(19) United States
(12) Patent Application Publication
(10) Pub. No.: US 2011/0054179 A1
(43) Pub. Date: Mar. 3, 2011

(54) MMP-2 AND/OR MMP-9 INHIBITOR

(76) Inventors: Shinya Minatoguchi, Gifu (JP); Yasushi Ohno, Gifu (JP); Youichi Yabuuchi, Tokushima (JP); Kounori Kotosai, Osaka (JP); Hisashi Nagamoto, Osaka (JP)

(21) Appl. No.: 12/922,374

(22) PCT Filed: Mar. 13, 2009
(86) PCT No.: PCT/JP2009/055545

§ 371 Date: Nov. 16, 2010

(30) Foreign Application Priority Data
Mar. 14, 2008 (JP) 2008-065455

(51) Int. Cl. C07D 417/04

(52) U.S. Cl. 546/270.4

(57) ABSTRACT

The present invention provides a highly safe pharmaceutical preparation effective for diseases caused by MMP-2 and/or MMP-9. The pharmaceutical preparation contains, as an active ingredient, at least one member selected from the group consisting of thiazole derivatives represented by Formula (1):

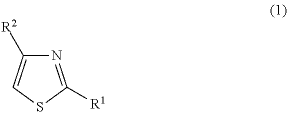

wherein R¹ represents a phenyl group that may have 1 to 3 lower alkoxy groups as substituents on the phenyl ring, and R² represents a pyridyl group that may have 1 to 3 carboxyl groups as substituents on the pyridine ring, and salts thereof. Such thiazole derivatives have MMP-2 and/or MMP-9 inhibitory activity.

### TECHNICAL FIELD

The present invention relates to a matrix metalloprotease (hereinafter referred to as “MMP”)-2 and/or MMP-9 inhibitor.

### BACKGROUND ART

The matrix metalloprotease is a collective term for extracellular matrix-degrading enzymes that contains a zinc (II) ion in their active site. The extracellular matrix turnover is mainly controlled by the balance between MMPs and a tissue inhibitor of metalloprotease (TIMP) specific to the MMPs.

MMPs consist of ten or more enzyme species, such as collagenase (MMP-1 and MMP-8), stromelysin (MMP-3), gelatinase (MMP-2 and MMP-9), etc., and they are produced in many types of cells.

Among the MMPs, the gelatinase group (MMP-2 and MMP-9) is known not only to possess gelatin-degrading activity, but also to digest type-IV collagen, fibronectin, vitronectin, etc.

However, highly safe pharmaceutical preparations that inhibit MMP-2 and/or MMP-9, and are effective as the treatment of the diseases caused by these MMPs, have not yet been launched.

Thiazole derivatives represented by the formula:

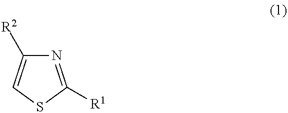

wherein R¹ represents a phenyl group that may have 1 to 3 lower alkoxy groups as substituents on the phenyl ring, and R² represents a pyridyl group that may have 1 to 3 carboxyl groups as substituents on the pyridine ring, or salts thereof are known to have inhibitory action against superoxide (O₂⁻) production, cytokine production, and adhesion of the cells, in addition to the beneficial action on chronic obstructive pulmonary disease (for example, Japanese Unexamined Patent Publication No. H5-51318, Japanese Unexamined Patent Publication No. H10-152437, Japanese Unexamined Patent Publication No. 2003-104890, etc.).

However, it is completely unknown at this moment that the thiazole derivatives represented by the above formula (I) or salts thereof exert MMP-2 and/or MMP-9 inhibitory activity that is completely different from the pharmacological activities listed above.

### DISCLOSURE OF THE INVENTION

### Problems to be Solved by the Invention

An object of the present invention is to provide a highly safe pharmaceutical preparation effective for diseases caused by MMP-2 and/or MMP-9.

### Means for Solving the Problems

The present inventors conducted extensive research to achieve the above object, and found that the thiazole derivatives, which are disclosed in the above Patent Publications as having O₂⁻ production inhibitory activity, cytokine production inhibitory activity, adhesion inhibitory activity, and chronic obstructive pulmonary disease treatment activity, also have MMP-2 and/or MMP-9 inhibitory activity, which cannot be expected by a person skilled in the art from the pharmacological activities listed above. The present invention has been accomplished based on such findings.

The present invention provides an MMP-2 and/or MMP-9 inhibitor according to the following Items 1 to 4.

Item 1. An MMP-2 and/or MMP-9 inhibitor comprising, as an active ingredient, at least one member selected from the group consisting of thiazole derivatives represented by Formula (1):

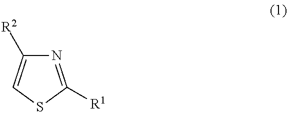

wherein R¹ represents a phenyl group that may have 1 to 3 lower alkoxy groups as substituents on the phenyl ring, and R² represents a pyridyl group that may have 1 to 3 carboxyl groups as substituents on the pyridine ring, and salts thereof.

Item 2. The MMP-2 and/or MMP-9 inhibitor according to Item 1, wherein the thiazole derivative is 6-[2-(3,4-diethoxyphenyl)thiazol-4-yl]pyridine-2-carboxylic acid or a salt thereof.

Item 3. The MMP-2 and/or MMP-9 inhibitor according to Item 1 or 2, for use in the treatment of fibrosis.

Item 4. The MMP-2 and/or MMP-9 inhibitor according to Item 1 or 2, for use in the treatment of pulmonary emphysema. The thiazole derivatives represented by Formula (I) of the present invention is a known compound, which may be produced by, for example, the method disclosed in Japanese Unexamined Patent Publication No. H5-51318.

Specific examples of the groups shown in Formula (I) are respectively as follows.

Examples of phenyl groups that may have 1 to 3 lower alkoxy groups as substituents on the phenyl ring include phenyl groups that may have 1 to 3 straight- or branched-chain alkoxy groups having 1 to 6 carbon atoms as substituents on the phenyl ring, such as phenyl, 2-methoxyphenyl, 3-methoxyphenyl, 4-methoxyphenyl, 2-ethoxyphenyl, 3-ethoxyphenyl, 4-ethoxyphenyl, 4-isopropoxyphenyl, 4-pentyloxyphenyl, 3-ethoxy-4-methoxyphenyl, 4-hexyloxyphenyl, 3,4-dimethoxyphenyl, 3,4-diethoxyphenyl, 2,3-dimethoxyphenyl, 2,6-dimethoxyphenyl, 3-propoxy-4-methoxyphenyl, 3,5-dimethoxyphenyl, 3,4-dipentyloxyphenyl, 3,4,5-trimethoxyphenyl, 3-methoxy-4-ethoxyphenyl, and the like.

Examples of pyridyl groups that may have 1 to 3 carboxyl groups as substituents on the pyridine ring include pyridyl, 2-carboxypyridyl, 3-carboxypyridyl, 4-carboxypyridyl, 2,3-dicarboxylpyridyl, 3,4-dicarboxylpyridyl, 2,4-dicarboxylpyridyl, 3,5-dicarboxylpyridyl, 3,6-dicarboxylpyridyl, 2,6-dicarboxylpyridyl, 2,4,6-tricarboxylpyridyl, and the like.

Among the thiazole derivatives represented by Formula (1) of the present invention, the compounds that have a basic group easily react with a usual pharmacologically acceptable acid to form a salt. Examples of such acids include inorganic acids, such as sulfuric acid, nitric acid, hydrochloric acid, phosphoric acid, hydrobromic acid, and the like; and organic acids, such as acetic acid, p-toluenesulfonic acid, ethanesulfonic acid, oxalic acid, maleic acid, fumaric acid, malic acid, tartaric acid, citric acid, succinic acid, benzoic acid, and the like.

Among the thiazole derivatives represented by Formula (1) of the present invention, the compounds that have an acidic group easily react with a pharmaceutically acceptable basic compound to form a salt. Examples of such basic compounds include sodium hydroxide, potassium hydroxide, calcium hydroxide, sodium carbonate, potassium hydrogencarbonate, and the like.

The thiazole derivatives of the present invention have optical isomers.

The compounds represented by Formula (1) are usually used in the form of a general pharmaceutical preparation. Such a pharmaceutical preparation may be prepared with commonly used diluents or excipients, such as fillers, extenders, binders, humectants, disintegrants, surfactants, lubricants, and the like.

The pharmaceutical preparation may take various forms, depending on the treatment purpose. Typical examples of such forms include tablets, pills, powders, solutions, suspensions, emulsions, granules, capsules, suppositories, injections (solutions, suspensions, etc.), inhalations, and the like.

In the preparation of the pharmaceutical preparation in tablet form, various kinds of carriers that are well known in the art may be used. Examples of such carriers include excipients, such as lactose, sucrose, sodium chloride, glucose, urea, starch, calcium carbonate, kaolin, crystalline cellulose, silicic acid, and the like; binders, such as water, ethanol, propanol, simple syrup, glucose solutions, starch solutions, gelatin solutions, carboxymethyl cellulose, shellac, methylcellulose, potassium phosphate, polyvinylpyrrolidones, and the like; disintegrants, such as dry starch, sodium alginate, agar powder, laminaran powder, sodium hydrogencarbonate, calcium carbonate, polyoxyethylene sorbitan fatty acid esters, sodium lauryl sulfate, stearic acid monoglyceride, starch, lactose, and the like; disintegration inhibitors, such as sucrose, stearin, cacao butter, hydrogenated oil, and the like; absorption promoters, such as quaternary ammonium base, sodium lauryl sulfate, and the like; moisturizers, such as glycerol, starch, and the like; adsorbents, such as starch, lactose, kaolin, bentonite, colloidal silica, and the like; and lubricants, such as purified talc, stearate, boric acid powder, polyethylene glycols, and the like. The tablets may further be, as necessary, coated with a common coating materials to obtain, for example, sugar-coated tablets, gelatin-coated tablets, enteric coated tablets, film coated tablets, or double- or multiple-layer tablets.

In the preparation of the pharmaceutical preparation in pill form, various kinds of carriers that are well known in the art may be used. Examples of the carriers include excipients, such as glucose, lactose, starch, cacao butter, hydrogenated vegetable oil, kaolin, talc, and the like; binders, such as gum arabic powder, tragacanth powder, gelatin, ethanol, and the like; and disintegrants, such as laminaran, agar, and the like.

In the preparation of the pharmaceutical preparation in suppository form, various kinds of carriers that are well known in the art may be used. Examples of the carriers include polyethylene glycols, cacao butter, higher alcohols, higher alcohol esters, gelatin, semi-synthetic glycerides, and the like.

Capsules may be prepared, in accordance with a known method, by mixing a usual active ingredient compound with the various kinds of carriers exemplified above, and filling the mixture in hard gelatin capsules, elasticity capsules, etc.

In the preparation of the pharmaceutical preparation as injections, it is preferable that the solutions, emulsions, and suspensions are sterilized and made isotonic with blood. In the preparation of the pharmaceutical preparations in such forms, any diluents conventionally used in the art may be utilized. Examples of such diluents include water, ethyl alcohol, macrogol, propylene glycol, ethoxylated isostearyl alcohol, polyoxylated isostearyl alcohols, polyoxyethylene sorbitan fatty acid esters, and the like. In this case, the pharmaceutical preparations may contain salt, glucose, or glycerol in an amount sufficient to make the resulting preparations isotonic. Further, usual solubilizers, buffers, soothing agents, etc. may further be added thereto.

In addition, colorants, preservatives, flavors, flavorings, sweeteners, etc., and other drugs may further be, as necessary, added to the pharmaceutical preparations.

Inhalation preparations may be prepared in accordance with a known method. Specifically, inhalation preparations may be prepared by making an active ingredient compound into a powder form or a liquid form, adding the obtained powder or liquid to an inhalation propellant and/or carrier, and filling the mixture into a suitable inhalation container. When the active ingredient compound is in powder form, a general mechanical powder inhalator is used. When the active ingredient compound is in liquid form, an inhaler, such as a nebulizer etc., may be used. As inhalation propellants, known inhalations may be used. Examples thereof include fluorocarbons, such as flon 11, flon 12, flon 21, flon 22, flon 113, flon 114, flon 123, flon 142c, flon 134a, flon 227, flon C318, 1,1,1,2-tetrafluoroethane, etc.; hydrocarbons, such as propane, isobutane, n-butane, etc.; ethers, such as diethyl ether, etc.; and compressed gases, such as nitrogen gas, carbon dioxide gas, etc.

Conventionally used surfactants, oils, seasonings, cyclodextrin or its derivatives, etc. may further be suitably added to the inhalation preparation of the present invention, if necessary. Examples of such surfactants include oleic acid, lecithin, diethylene glycol dioleate, tetrahydrofurfuryl oleate, ethyl oleate, isopropyl myristate, glyceryl trioleate, glyceryl monolaurate, glyceryl monooleate, glyceryl monostearate, glyceryl monoricinoleate, cetyl alcohol, stearyl alcohol, polyethylene glycol 400, cetylpyridinium chloride, sorbitan trioleate (trade name: span 85), sorbitan monooleate (trade name: span 80), sorbitan monolaurate (trade name: span 20), polyoxyethylene hydrogenated castor oil (trade name: HCO-60), polyoxyethylene (20) sorbitan monolaurate (trade name: Tween 20), polyoxyethylene (20) sorbitan monooleate (trade name: Tween 80), lecithin derived from natural sources (trade name: Epikuron), oleyl polyoxyethylene (2) ether (trade name: Brij 92), stearyl polyoxyethylene (2) ether (trade name: Brij 72), lauryl polyoxyethylene (4) ether (trade name: Brij 30), oleyl polyoxyethylene (2) ether (trade name: Genapol 0-020), block copolymer of oxyethylene and oxypropylene (trade name: Synperonic), etc. Examples of oils include corn oil, olive oil, cottonseed oil, sunflower seed oil, etc.

When preparing the active ingredient compound of the present invention in liquid form, the active ingredient compound may be, for example, dissolved in a carrier in liquid form.

Examples of such carriers in liquid form include water, salt water, organic solvents, etc. Among these, water is preferable. In dissolution, surfactants, such as polyethylene glycol having a molecular weight of 200 to 5000, polyoxyethylene (20) sorbitan monooleate, etc.; sodium carboxymethylcellulose, methylcellulose, polyvinylpyrrolidone, polyvinyl alcohol, etc. may be suitably added thereto.

When preparing the active ingredient compound of the present invention in powder form, the pulverization may be carried out in accordance with a known method. For example, it is preferable that the active ingredient compound is pulverized with lactose, starch, etc., and stirred to form a uniformly mixed powder.

The amount of the active ingredient compound contained in the therapeutic preparations of the present invention is not limited, and may be adjusted in a wide range. It is usually preferable that the preparation composition contains about 1 to about 70% by weight of the active ingredient compound.

Administration methods of the therapeutic preparations of the present invention are not specifically limited, and may be administered depending on the form of the drug, the age, sex, and other conditions of the patient, the disease conditions of patient, and the like. For example, tablets, pills, solutions, suspensions, emulsions, granules, and capsules are orally administered. Injection preparations are intravenously administered singly or in combination with reinfusions, such as glucose, amino acid, etc.; and if necessary, the injection preparations are administered singly intramuscularly, intracutaneously, subcutaneously, or intraperitoneally. Suppositories are intrarectally administered. Inhalation preparations are inhaled into the oral cavity.

The dosage of the therapeutic preparations of the present invention is suitably selected according to the usage, the age, sex and other conditions of the patient, the disease conditions of the patient, and the like, but is usually about 0.2 to about 200 mg/kg of body weight per day in terms of the usual active ingredient compound.

### EFFECT OF THE INVENTION

The present invention provides a highly safe pharmaceutical preparation that is effective as the treatment of the diseases caused by MMP-2 and/or MMP-9.

The MMP-2 and/or MMP-9 inhibitor of the present invention selectively inhibits MMP-2 and/or MMP-9. More specifically, the MMP-2 and/or MMP-9 inhibitor of the present invention inhibits the expression of MMP-2 and/or MMP-9. Examples of effective indications of the MMP-2 and/or MMP-9 inhibitor of the present invention include RAs and bone diseases, such as rheumatoid arthritis, arthritis, arthrosis, disease of bone, osteoporosis, bone injury, osteoarthritis, bone dysbolism, etc.; inflammations, such as Crohn's disease, eye inflammation, inflammatory bowel disease, anaphylaxis, irritable bowel syndrome, bacterial infection, periodontal disease, otitis, ulcer, ulcerative colitis, mucitis, pneumonia, abdominal inflammation, cystitis, etc.; cancer diseases, such as a lymphoma, gastric tumor, cancerous pleural effusion, cancerous ascites, solid carcinoma, melanoma, bone metastases, alimentary canal tumor, esophageal cancer, glioma, renal cell cancer, astrocytoma, prostate tumor, multiple myeloma, metastasis, head and neck tumor, sarcoma, breast cancer, brain tumor, lung tumor, non-small cell lung cancer, eye cancer, ovarian tumor, glioblastoma, pancreas tumor, etc.; blood and endocrine diseases, such as type 2 diabetes mellitus, insulin-independent diabetes mellitus, hyperphosphatemia, myelodysplastic syndrome, diabetes mellitus, leukaemia, etc.; cardiovascular diseases, such as congestive heart failure, hypertension, atherosclerosis, acute coronary-artery syndrome, vascularization disorder, restenosis, cardiac infarction, cardiovascular disorders, cardiopathy, cardiac insufficiency, aortic aneurysm, diabetic nephropathy, cerebrovascular ischemia, and cerebral infarction, etc.; eye and neurological disorders, such as age-related macular degeneration, corneal injury, corneal ulcer, infectious diseases in the ophthalmologic field, dry eye sensation, eye diseases, neurological disease, neurodegenerative disease, multiple sclerosis, diabetic retinopathy, retinal macular degeneration, pterygium, lacrimal gland disease, etc.; infectious diseases, such as HIV infection, Clostridium botulinum infection, oral infection, respiratory tract infection of bacteria, Plasmodium falciparum infection, Clostridium tetani infection, septic fever, septic shock, etc.; respiratory diseases, such as asthma, respiratory system disease, pulmonary emphysema, etc.; skin diseases, such as atopic dermatitis, Kaposi's sarcoma, psoriasis, acne, rosacea, skin burns, skin disease, scar tissue, chronic skin ulcer, etc.; and other diseases, such as Alzheimer's disease, proteinuria, epilepsy, graft versus host disease, chemotherapy induction injury, kidney disease, fibrosis, wound healing, diabetic complications, toxin poisoning, endotoxic shock, brain damage, lung damage, anemia, pain, etc.

The MMP-2 and/or MMP-9 inhibitor of the present invention exerts significantly high therapeutic efficacy particularly to pulmonary fibrosis and pulmonary emphysema.

### BEST MODE FOR CARRYING OUT THE INVENTION

Formulation Examples and Test Examples are given below. Hereinafter, “Compound A” refers to 6-[2-(3,4-diethoxyphenyl)thiazol-4-yl]pyridine-2-carboxylic acid.

### Formulation Example 1

| | | |
|---|---|---|
| Compound A | 150 | g |
| Avicel (trademark, produced by Asahi Kasei Corporation) | 40 | g |
| Cornstarch | 30 | g |
| Magnesium Stearate | 2 | g |
| Hydroxypropylmethylcellulose | 10 | g |
| Polyethylene Glycol 6000 | 3 | g |
| Castor Oil | 40 | g |
| Ethanol | 40 | g |

Compound A, Avicel, cornstarch and magnesium stearate were mixed and ground. The resulting mixture was shaped into tablets by using a pounder (R 10 mm) for sugar coating. The obtained tablets were coated with a film coating agent containing hydroxypropylmethylcellulose, polyethylene glycol 6000, castor oil and ethanol. Thereby, film-coated tablets were prepared.

### Formulation Example 2

| | | |
|---|---|---|
| Compound A | 150 | g |
| Citric Acid | 1.0 | g |
| Lactose | 33.5 | g |
| Dicalcium Phosphorate | 70.0 | g |
| Pluronic F-68 | 30.0 | g |
| Sodium Lauryl Sulfate | 15.0 | g |
| Polyvinylpyrrolidone | 15.0 | g |
| Polyethylene Glycol (Carbowax 1500) | 4.5 | g |
| Polyethylene Glycol (Carbowax 6000) | 45.0 | g |
| Cornstarch | 30.0 | g |
| Dry Sodium Stearate | 3.0 | g |
| Dry Magnesium Stearate | 3.0 | g |

| | |
|---|---|
| Ethanol | q.s. |

Compound A, citric acid, lactose, dicalcium phosphorate, Pluronic F-68 and sodium lauryl sulfate were mixed together.

The resulting mixture was sieved through a No. 60 screen. The sieved mixture was wet granulated with an alcohol solution containing polyvinyl pyrrolidone, Carbowax 1500 and Carbowax 6000. Alcohol was added, as necessary, to the resulting wet granulated powder, which was then converted into a paste-like mass. Subsequently, cornstarch was added to the obtained paste-like mass, and a mixing operation was conducted thereto until uniform particles were formed. The resulting particle mixture was sieved through a No. 10 screen, placed on a tray, and dried in an oven at 100° C. for 12 to 14 hours. The dried particles were sieved through a No. 16 screen. Thereafter, dry sodium lauryl sulfate and dry magnesium stearate were added to the obtained sieved particles, and mixed together. Then, the resulting mixture was compressed into core tablets having a desired shape by means of a tablet machine.

The obtained core tablets were treated with varnish, and talc was sprayed thereon for preventing moisture absorption. An undercoat layer was applied on the surfaces of the resulting core tablets. Then, varnish was applied to the undercoat layer a sufficient number of times so as to prepare the tablets for internal use. To make the resulting coated tablets completely round and smooth, an undercoat layer and a smooth layer were further applied thereon. Thereafter, a colored coating was applied so that the tablet surface had a desired color. The coated tablets were dried and then polished to thereby obtain tablets having uniform gloss.

### Formulation Example 3

| | | |
|---|---|---|
| Compound A | 5 | g |
| Polyethylene Glycol (molecular weight: 4000) | 0.3 | g |
| Sodium Chloride | 0.9 | g |
| Polyoxyethylene Sorbitan Monooleate | 0.4 | g |
| Sodium Metabisulphite | 0.1 | g |
| Methylparaben | 0.18 | g |
| Propylparaben | 0.02 | g |
| Distilled Water for Injection | 10.0 | ml |

The above-listed parabens, sodium metabisulfite and sodium chloride were dissolved in about a half volume of the above-mentioned distilled water at 80° C. with stirring. The resulting solution was cooled to 40° C. Then, Compound A, subsequently polyethylene glycol and polyoxyethylene sorbitan monooleate were dissolved in the solution. To the obtained solution was added another half of the volume of the distilled water for injection, so as to adjust the solution to have a final volume. The thus-obtained solution was sterilized by subjecting to sterilizing filtration using an appropriate filter paper. Thereby, an injection was prepared.

### Test Example

### Rabbit Model of Elastase-Induced Lung Injury

### Test Procedures:

Rabbits were divided into three groups (n=10 animals/group). Two hundred U/kg of porcine pancreatic elastase (PPE) was intratracheally administered into the lungs of the animals in the Vehicle and the Compound A group. The animals in the Sham group was intratracheally administered the same volume of saline instead of PPE. The rabbits were dissected 28 days after PPE administration. The lung tissue of each rabbit was fixed in formalin to prepare histological sections thereof. Two hours prior to the administration of PPE, Vehicle (0.5% tragacanth) or Compound A (10 mg/kg) was orally administered to the rabbits in the Vehicle and the Compound A group, respectively; from the next day, the oral administration of Vehicle or Compound A was continued once a day, 5 days a week until the end of the experiment.

The histological sections were immunohistochemically stained using respective antibodies against MMP-2 or MMP-9. Then, under the microscope, the extent of MMP-2 and MMP-9 expression was evaluated, and expressed as a score. The extent of the fibrotic changes in the airway subepithelial region and the extent of alveolar destruction were also observed.

### Test Result

Table 1 summarizes the MMP expression in the lungs of the animals in the respective groups. The Vehicle group demonstrated significantly higher MMP-2 and MMP-9 expression scores (both of them are p<0.01) compared to those of the Sham group. In addition, the thickened airway subepithelial layer and fibrotic changes was also observed in the lungs of the Vehicle group. Contrary, the MMP-2 and MMP-9 expression scores of the Compound A group were significantly lower than those of the Vehicle group (MMP-2: p<0.05; and MMP-9: p<0.01). Further, the thickening of the airway subepithelial layer resulted from fibrotic changes was alleviated, and the alveolar destruction was significantly suppressed. Table 2 shows the mean linear intercept, a typical parameter of alveolar space enlargement. Based on the results described above, it is demonstrated that Compound A significantly suppresses the MMP-2 and MMP-9 expression.

TABLE 1
MMP Expression Score Evaluated Using Pathological Specimens

| | | Score (Average ± Standard deviation) | |
|---|---|---|---|
| Group | Number | MMP-2 | MMP-9 |
| Sham Group | 10 | 0.58 ± 0.04 | 0.73 ± 0.06 |
| Vehicle Group | 10 | 2.02 ± 0.18 ** | 2.40 ± 0.03 ** |
| Compound A Group | 10 | 1.49 ± 0.12 # | 1.81 ± 0.03 ## |

The differences between the Sham and the Vehicle groups, and between the Vehicle and the Compound A groups were analyzed by multiple comparison tests. In Table 1, **: p<0.01, vs. Sham, #: p<0.05, vs. Vehicle, and ##: p<0.01, vs. Vehicle.

TABLE 2
Alveolar Mean Linear Intercept

| | | Alveolar Mean Linear Intercept |
|---|---|---|
| Group | Examples | (Average ± Standard deviation) |
| Sham Group | 10 | 48.7 ± 1.0 μm |
| Vehicle Group | 10 | 104.5 ± 6.2 μm ** |
| Compound A Group | 10 | 72.8 ± 2.6 μm ## |

The Comparisons of mean linear intercept between the Sham and the Vehicle groups, and between the Vehicle and the Compound A groups were performed by multiple comparison tests. In Table 2, **: p<0.01, vs. Sham, ##: p<0.01, vs. Vehicle.

### Discussion on the Relation Between Fibrosis and MMP Expression

The fibrosis occurred in various tissues is a serious disease with poor prognosis. The main histological characteristics thereof are the injury of endothelial and epithelial cells; the inflammation consisting of infiltration of neutrophils, macrophages and lymphocytes; the proliferation of fibroblasts; and the excessive synthesis and deposition of extracellular matrix (ECM) components, such as collagen. In particular, the excessive synthesis and deposition of ECM is considered to be caused by disruption of the balance between MMPs, enzymes degrading the ECM selectively, and TIMP (tissue inhibitor of metalloprotease), a substance controlling the ECM activity in vivo. However, the details of the mechanism thereof remain unclear. On the other hand, it was reported that the levels of MMP (in particular, MMP-2 and MMP-9) expression elevated in the lung tissue and bronchoalveolar lavage fluid in the patients with lung fibrosis, and similar results were also reported in animal models of lung fibrosis. Other reports demonstrated that in the animal model of bleomycin- or asbestos-induced lung fibrosis, the administration of MMP inhibitors, such as batimastat or GM6001, suppressed the increase in MMP activity and the number of white blood cell infiltrated in the bronchoalveolar lavage fluid; as a result, the lung fibrosis was suppressed in the histological and biochemical aspects. These results illustrates that the increase in the MMP enzyme activity or the amount of expression induces the fibrotic changes in the tissues. Based on this evidence, it is likely that suppressing the MMP activity in the tissues results in inhibiting the fibrosis of the tissues.

Taken together, it is strongly suggested that the compound that suppresses the MMP expression may inhibit tissue fibrosis. From viewpoint of the foregoing results, which illustrate that Compound A inhibits the expression of both MMP-2 and MMP-9 in lung tissue, in addition to the suppression in alveolar destruction and the fibrotic changes in the airway subepithelial, it is clear that the compounds represented by Formula (1) of the present invention or salts thereof is remarkably effective as a therapeutic preparation for fibrosis, in particular, for a lung fibrosis, and/or pulmonary emphysema.

1. An MMP-2 and/or MMP-9 inhibitor comprising, as an active ingredient, at least one member selected from the group consisting of thiazole derivatives represented by Formula (1): 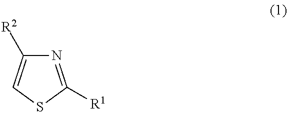
wherein R¹ represents a phenyl group that may have 1 to 3 lower alkoxy groups as substituents on the phenyl ring and R² represents a pyridyl group that may have 1 to 3 carboxyl groups as substituents on the pyridine ring, and salts thereof.

2. The MMP-2 and/or MMP-9 inhibitor according to claim 1, wherein the thiazole derivative is 6-[2-(3,4-diethoxyphenyl)thiazol-4-yl]pyridine-2-carboxylic acid or a salt thereof.

3. The MMP-2 and/or MMP-9 inhibitor according to claim 1 or 2, for use in the treatment of fibrosis.

4. The MMP-2 and/or MMP-9 inhibitor according to claim 1 or 2, for use in the treatment of pulmonary emphysema.
